(19) United States
(12) Patent Application Publication
(10) Pub. No.: US 2012/0093938 A1
(43) Pub. Date: Apr. 19, 2012

(54) ORALLY DISINTEGRATING TABLETS COMPRISING DIPHENHYDRAMINE

(75) Inventors: Gopi M. VENKATESH, Vandalia, OH (US); Jin-Wang LAI, Springborro, OH (US); Phillip PERCEL, Troy, OH (US); Craig KRAMER, New Lebanon, OH (US)

(73) Assignee: EURAND INC., Vandalia, OH (US)

(21) Appl. No.: 13/183,125

(22) Filed: Jul. 14, 2011

Related U.S. Application Data
(63) Continuation of application No. 12/331,963, filed on Dec. 10, 2008, now abandoned.
(60) Provisional application No. 61/012,531, filed on Dec. 10, 2007.

(51) Int. Cl. A61K 9/14; A61K 31/485; A61K 31/167; A61K 31/192; A61K 31/5415; A61K 31/616; A61K 31/635; A61K 31/407; A61K 31/405; A61K 31/196; A61P 25/16; A61P 31/00; A61P 29/00; A61P 25/00; A61P 1/08; A61J 3/10; A61K 31/135

(52) U.S. Cl. 424/495; 514/648; 424/490; 514/282; 514/629; 514/570; 514/226.5; 514/162; 514/158; 514/411; 514/420; 514/567; 424/494; 424/497; 264/109; 264/134

(57) ABSTRACT

The compositions of the present invention comprise a therapeutically effective amount of particles consisting of diphenhydramine or pharmaceutically acceptable salts thereof, optionally in combination with another drug such as pseudoephedrine, or phenylephrine and hydrocodone, in combination with rapidly-dispersing microgranules comprising a disintegrant and a sugar alcohol and/or a saccharide. These compositions are useful in treating the symptoms of one or more diseases or conditions in which diphenhydramine (alone or in combination with one or two other drugs) is a therapeutically effective, e.g. allergic rhinitis, sinusitis, upper respiratory tract infections, motion sickness, Parkinson's disease, insomnia, the common cold, and nighttime pain management, particularly for subjects or patients with dysphagia, and people ‘on the move’.

### CROSS-REFERENCE TO RELATED APPLICATIONS

The present application claims priority to U.S. Provisional Application No. 61/012,531 filed Dec. 10, 2007, the disclosure of which is herein incorporated by reference in its entirety for all purposes.

### TECHNICAL FIELD

This invention relates to immediate release (IR), orally disintegrating tablet (ODT) compositions comprising diphenhydramine or a pharmaceutically acceptable salt thereof, or a combination of diphenhydramine with hydrocodone, pseudoephedrine and/or phenylephrine, useful for the treatment of symptoms of one or more of allergic rhinitis, sinusitis, upper respiratory tract infections, motion sickness, and Parkinson's disease, and to induce sleep or relieve symptoms associated with the common cold or a combination of diphenhydramine with non-opioid analgesics for pain management (e.g. at night).

### BACKGROUND OF THE INVENTION

Dysphagia, or difficulty in swallowing due to fear of choking, is common among all age groups. For example, it is observed in about 35% of the general population, as well as an additional 30-40% of elderly institutionalized patients and 18-22% of all persons in long-term care facilities, many of whom are required to consume medications on a regular basis to maintain their quality of life. Diphenhydramine by itself, or in combination with hydrocodone bitartrate, pseudoephedrine HCl and/or phenylephedrine HCl is generally available as a tablet or a capsule for oral administration taken 2-4 times a day, or as directed. The need for multiple doses leads to poor or even non-compliance thus has a negative impact on the efficacy of the treatment, especially in children who are unwilling or have difficulty in swallowing capsules or tablets three to four times a day. In addition, some pediatric, geriatric, and psychiatric patient populations exhibit “cheeking” behavior (i.e., holding the oral dosage form in the cheek) to avoid swallowing the medication. Accordingly, ODT formulation would be desirable to improve patient compliance, particularly among elderly, pediatric and institutionalized patients, because ODT formulations are easier to swallow and prevent “cheeking”. In particular, ODT formulations that can provide once-a-day dosing would be particularly desirable.

ODT formulations must be palatable, e.g. have acceptable organoleptic properties such as good taste and mouthfeel to maintain patient compliance or adherence to the dosing regimen, because ODT tablets are designed to disintegrate in the mouth of the patient. ODT compositions must also provide acceptable pharmacokinetic and bioavailability characteristics to provide the desired therapeutic effect. For bitter tasting drugs such as diphenhydramine, phenylephrine, pseudoephedrine, and hydrocodone, ODT formulations require the application of a taste-masking layer to the drug-containing particles to improve the organoleptic characteristics of the formulation. However, taste-masking can inhibit or delay drug release, thereby providing unacceptable pharmacokinetic properties. Conversely, components of the formulation that promote rapid release may result in undesirable taste or mouthfeel properties. Accordingly, an acceptable ODT formulation must balance these contradictory characteristics in order to provide a palatable (e.g., taste-masked), fast disintegrating composition with acceptable pharmacokinetics.

### SUMMARY OF THE INVENTION

The present invention is directed to an orally disintegrating tablet (ODT) composition comprising a therapeutically effective amount of diphenhydramine-containing particles coated with a taste-masking layer, at least one disintegrant, and at least one sugar alcohol and/or at least one saccharide; wherein the diphenhydramine-containing particles comprise diphenhydramine; the taste-masking layer comprises a water-insoluble polymer.

In one embodiment of the ODT compositions of the present invention, the diphenhydramine-containing particles are drug-layered beads comprising an inert core coated with a diphenhydramine-containing layer.

In another embodiment of the ODT compositions of the present invention, the taste-masking layer comprises a water-insoluble taste-masking polymer or a water-insoluble taste-masking polymer in combination with a water-soluble or gastrosoluble pore former.

In yet another embodiment, the present invention is directed to a method of preparing the ODT compositions of the present invention comprising preparing particles comprising diphenhydramine; coating the diphenhydramine-containing particles with a taste-masking layer; preparing granules comprising a disintegrant in combination with a sugar alcohol and/or a saccharide; mixing the diphenhydramine-containing particles coated with a taste-masking layer with the disintegrant-containing granules and optionally other pharmaceutically acceptable ingredients; and compressing the mixture into tablets.

In still another embodiment, the present invention is directed to a method of treating the symptoms of one or more diseases or conditions in which diphenhydramine is therapeutically effective, including but not limited to allergic rhinitis, sinusitis, upper respiratory tract infections, motion sickness, Parkinson's disease, insomnia, and the common cold, comprising administering the ODT composition of the present invention.

In yet another embodiment, the present invention is directed to a method of treating pain (e.g., treatment of night pain for better sleep management) by oral administration of a combination ODT product comprising taste-masked diphenhydramine and acetaminophen at 25 mg and about 250 mg, respectively, wherein the analgesic acetaminophen is taste-masked by solvent coacervation in cyclohexane using a water-insoluble ethylcellulose as a taste-masking coating material.

### BRIEF DESCRIPTION OF THE DRAWINGS

FIG. 1 shows variations in friability as a function of compression force at tablet weights of 400-mg, 450-mg and 500-mg for ODT formulations comprising diphenhydramine microparticles of Example 1.

FIG. 2 shows sampling locations in a V-blender for blend homogeneity testing.

FIG. 3A shows variations in tablet hardness as a function of compression force, and FIG. 3B shows variations in tablet friability as a function of hardness for ODT tablets of Example 1F, at various press turntable speeds.

FIG. 4 shows dissolution profiles for diphenhydramine hydrochloride and phenylephrine from ODT formulations of Example 3D.

### DETAILED DESCRIPTION OF THE INVENTION

The term “drug”, “active” or “active pharmaceutical ingredient” as used herein includes any pharmaceutically acceptable and therapeutically effective compound (e.g., diphenhydramine), as well as pharmaceutically acceptable salts, stereoisomers and mixtures of stereoisomers, solvates (including hydrates), and/or esters thereof. Similarly, any reference to specific drugs (e.g., diphenhydramine, pseudoephedrine, phenylephrine, hydrocodone, acetaminophen, aspirin, etodolac, diclofenac potassium, ibuprofen, ketoprofen, meloxicam, celecoxib, endomethacin, sulindac, etc.) includes salts, stereoisomers and mixtures of stereoisomers, solvates (including hydrates), and/or esters thereof, unless expressly stated otherwise.

Suitable salts include pharmaceutically acceptable acid'addition salts such as hydrochloric, hydrobromic, hydriodic, nitric, sulfuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used, e.g. acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, isobutyrate, phenylbutyrate, α-hydroxybutyrate, butyne-1,4-dicarboxylate, hexyne-1,4-dicarboxylate, caprate, caprylate, cinnamate, citrate, formate, fumarate, glycolate, heptanoate, hippurate, lactate, malate, maleate, hydroxymaleate, malonate, mandelate, mesylate, nicotinate, isonicotinate, oxalate, phthalate, terephthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, p-toluenesulfonate, xylenesulfonate, tartrate, bitartrate and the like.

In one embodiment, the ODT compositions of the present invention comprise diphenhydramine hydrochloride. In another embodiment, the ODT compositions of the present invention comprise diphenhydramine hydrochloride, in combination with one or more of pseudoephedrine hydrochloride, phenylephrine hydrochloride, and hydrocodone bitartrate.

The terms “orally disintegrating tablet”, “orally dispersible tablet”, or “ODT” refer to a solid dosage form of the present invention, which disintegrates rapidly in the oral cavity of a patient after administration. The rate of disintegration can vary, but is faster than the rate of disintegration of conventional solid dosage forms (i.e., tablets or capsules) which are intended to be swallowed immediately after administration. ODT compositions of the present invention can contain pharmaceutically acceptable ingredients which swell, dissolve or otherwise facilitating the disintegration or dissolution of the ODT composition.

The term “unit dose” refers to a pharmaceutical composition containing an amount of drug intended to be administered to a patient in a single dose.

The term “about” in reference to numerical quantities includes “exactly” the numerical quantity, as well as values near the numerical quantity. For example, “about 60 second” includes 60 seconds, exactly, as well as values close to 60 seconds (e.g., 50 seconds, 55 seconds, 59 seconds, 61 seconds, 65 seconds, 70 seconds, etc.).

The term “substantially disintegrates” in reference to the ODT compositions of the present invention means the disintegration of the ODT largely into its constituent particles which were previously compressed into monolithic tablets. Similarly, the term “substantially dissolves” in reference to the ODT compositions of the present invention means that the percentage of “active” (e.g., diphenhydramine) released or dissolved from the ODT is at least about 50%, at least about 60%, at least about 70%, at least about 80%, at least about 90%, or about 100% of the diphenhydramine present in the ODT composition.

The term “microparticle” refers to a particle with an average particle size of not more than about 400 μm, in some embodiments not more than about 300 μm. The terms “particle”, “microparticle”, “granule” and “microgranule” are used interchangeably herein to refer to a particle with a mean particle size of not more than about 400 μm, irrespective of the composition of the particle. The term “microencapsulation” as used herein refers to drug-containing particles coated with a taste-masking layer, having a mean particle size of not more than about 400 μm.

Unless indicated otherwise, all percentages and ratios are calculated by weight. Unless indicated otherwise, all percentages and ratios are calculated based on the total composition.

The microparticles herein can be described as primary particles or secondary particles. Primary particles are unagglomerated, whereas secondary particles are agglomerated primary particles. Thus, primary particles are smaller than secondary particles.

In one embodiment, the present invention is directed to an orally disintegrating tablet (ODD composition comprising a therapeutically effective amount of diphenhydramine-containing particles coated with a taste-masking layer, and rapidly dispersing microgranules.

The diphenhydramine-containing particles include crystalline diphenhydramine, diphenhydramine granulated with one or more pharmaceutically acceptable excipients (e.g., fillers, binders; etc.), or inert cores layered with a diphenhydramine-containing coating. For example, crystalline diphenhydramine can include primary particles of crystalline diphenhydramine having an average particle size ranging from about 1-300 μm, including about 1-50 μm, about 1-100 μm, about 1-150 μm, about 1-200 μm, about 1-250 μm, about 50-100 μm, about 50-150 μm, about 50-200 μm, about 50-250 μm, about 50-300 μm, about 100-150 μm, about 100-200 μm, about 150-200 μm, about 150-250 μm, about 150-300 μm, about 200-250 μm, about 200-300 μm, or about 250-300 μm.

When the diphenhydramine-containing particles are granules, the diphenhydramine-containing granules comprise diphenhydramine crystals granulated with at least a film-forming binder. The film-forming binder can comprise any suitable binder used in granulation. Non-limiting examples of suitable film-forming binders include water-soluble, alcohol-soluble or acetone/water soluble binders, e.g. polyvinylpyrrolidone (PVP), corn starch, polyethylene oxide, polyethylene glycol, hydroxypropyl methylcellulose (HPMC), methylcellulose, or hydroxypropylcellulose (HPC). The amount of film-forming binder in the diphenhydramine-containing granules can range from about 0.5% to about 10%, including about 0.5%-1%, about 0.5%-2%, about 0.5%-5%, about 0.5%-7%, about 1%-2%, about 1%-5%, about 1%-7%, about 1%-10%, about 2%-5%, about 2%-7%, about 2%-10%, about 5%-7%, about 5%-10%, and about 7%-10%.

The diphenhydramine-containing granules of the present invention can also include other pharmaceutically acceptable ingredients, for example, fillers or diluents. Non-limiting examples of other pharmaceutically acceptable ingredients for the drug-containing granules include, for example, mannitol, lactose, microcrystalline cellulose, potassium sulfate, calcium phosphate, modified starch, and mixtures thereof. The amount of other pharmaceutically acceptable ingredients (e.g. fillers or diluents) in the diphenhydramine-containing granules can range from about 5%-80%, including about 5%-70%, about 5%-60%, about 5%-50%, about 5%-40%, about 5%-30%, about 5%-20%, about 5%-15%, about 5%-10%, about 10%-70%, about 10%-60%, about 10%-50%, about 10%-40%, about 10%-30%, about 10%-20%, about 10%-15%, about 20%-70%, about 20%-60%, about 20%-50%, about 20%-40%, about 20%-30%, about 20%-25%, about 30%-70%, about 30%-60%, about 30%-50%, about 30%-40%, about 30%-35%; about 40%-70%, about 40%-60%, about 40%-50%, about 40%-45%, about 50%-70%, about 50%-60%, about 50%-55%, about 60%-70%, or about 60%-65%.

In another embodiment, the drug-containing cores of the present invention can be in the form of diphenhydramine-layered beads comprising a core, e.g. a pharmaceutically acceptable sugar sphere or cellulose sphere (Celphere® or Cellets®), coated with a diphenhydramine-containing layer comprising diphenhydramine and a polymeric binder. Suitable polymeric binders include any of those disclosed herein, for example starches, modified celluloses (e.g., hydroxypropylcellulose, carboxymethylcellulose sodium), alginic acid, polyvinylpyrrolidone (povidone), and mixtures thereof. The amount of diphenhydramine in the diphenhydramine layer, and the thickness of the diphenhydramine layer can be modified to provide a therapeutically effective dose of diphenhydramine. In one embodiment, the diphenhydramine-containing layer comprises about 90%-99% diphenhydramine as a HCl salt, and about 1% to about 10% of a polymeric binder.

The diphenhydramine-containing particles of the ODT compositions of the present invention (e.g., crystalline diphenhydramine, granulated diphenhydramine, or diphenhydramine-layered beads) are coated with a taste-masking layer. The taste masking layer comprises a water-insoluble polymer, optionally in combination with a water-soluble or gastrosoluble pore former. Pore formers increase the release rate of the diphenhydramine through the taste-masking layer. Water-soluble pore formers dissolve readily in water or saliva, whereas gastrosoluble pore formers are insoluble in water and saliva, but are readily soluble under acidic conditions, such as those found in the stomach.

Non-limiting examples of suitable water-insoluble polymers include, e.g., ethyl cellulose, polyvinyl acetate (PVA), cellulose acetate (CA), cellulose acetate butyrate (CAB), and methacrylate copolymers available under the trade name “EUDRAGIT” (such as Eudragit® RL, Eudragit® RS, Eudragit® NE30D, etc.). Non-limiting examples of water-soluble pore-formers include, e.g. sodium chloride, sucrose, povidone, and mixtures thereof. Non-limiting examples of gastrosoluble pore-formers include, e.g. calcium carbonate, magnesium citrate, magnesium hydroxide, and mixtures thereof. Non-limiting examples of gastrosoluble pore-forming polymers include, e.g. Eudragit® E100/EPO, AEA® (polyvinylacetal diethylaminoacetate available from Sankyo Company Limited, Tokyo), and mixtures thereof. When a pore former is present in the taste-masking layer, the ratio of water-insoluble polymer to water-soluble or gastrosoluble pore-former varies from about 95/5 to about 50/50 by weight. The amount of the taste-masking coating ranges from about 5% to about 30% of the total weight of the taste-masked diphenhydramine-containing particles, or about 5%-25%, about 5%-20%, about 5%-15%, about 5%-10%, about 10%-30%, about 10%-25%, about 10%-20%, about 10%-15%, about 15%-30%, about 50%-25%, about 15%-20%, about 20%-30%, about 20%-25%, or about 25%-30%.

The ODT compositions of the present invention include rapidly dispersing granules comprising a disintegrant and a sugar alcohol and/or a saccharide. Non-limiting examples of suitable disintegrants for the rapidly dispersing granules can include disintegrants or so-called super-disintegrants, e.g. crospovidone (crosslinked PVP), sodium starch glycolate, crosslinked sodium carboxymethyl cellulose, low substituted hydroxypropylcellulose, and mixtures thereof. The amount of disintegrant in the rapidly dispersing granules can range from about 1%-10%, or about 5%-10% of the total weight of the rapidly dispersing granules, including all ranges and subranges therebetween.

Sugar alcohols are hydrogenated forms of carbohydrates in which the carbonyl group (i.e., aldehyde or ketone) has been reduced to a primary or secondary hydroxyl group. Non-limiting examples of suitable sugar alcohols for the rapidly dispersing granules of the ODT compositions of the present invention can include e.g. arabitol, isomalt, erythritol, glycerol, lactitol, mannitol, sorbitol, xylitol, maltitol, and mixtures thereof.

The term “saccharide” is synonymous with the term “sugars”, and includes monosaccharides such as glucose, fructose, lactose, and ribose; and disaccharides such as sucrose, lactose, maltose, trehalose, and cellobiose. Non-limiting examples of suitable saccharides for use on the compositions of the present invention include e.g. lactose, sucrose, maltose, and mixtures thereof.

In one embodiment, the rapidly dispersing granules comprise at least one disintegrant in combination with a sugar alcohol. In another embodiment, the rapidly dispersing granules comprise at least one disintegrant in combination with a saccharide. In yet another embodiment, the disintegrant-containing granules comprise at least one disintegrant in combination with a sugar alcohol and a saccharide.

The amount of sugar alcohol and/or saccharide in the rapidly dispersing granules ranges from about 99%-90%, or about 95%-90% of the total weight of the rapidly dispersing granules, including all ranges and subranges therebetween. In one embodiment, the average particle size of the primary particles of sugar alcohol and/or saccharide is 30 μm or less, for example about 1-30 μm, about 5-30 μm, about 5-25 μm, about 5-20 μm, about 5-15 μm, about 5-10 μm, about 10-30 μm, about 10-25 μm, about 10-20 μm, about 10-15 μm, about 15-30 μm, about 15-25 μm, about 15-20 μm, about 20-30 μm, about 20-25 μm, or about 25-30 μm.

Prior to coating with the taste-masking layer, the diphenhydramine-containing particles (e.g., crystalline or amorphous diphenhydramine, granulated diphenhydramine, or diphenhydramine-layered beads) generally have an average particle size of about 1-100 μm, in some embodiments about 1-50 μm or about 1-30 μm, or average particle sizes as disclosed elsewhere herein. After coating with the taste-masking layer, the taste-masked diphenhydramine-containing particles generally have an average particle size of less than about 400 μm. If the average particle size is greater than about 400 μm, the disintegrated ODT can have an unpleasant “gritty” texture in the mouth of the patient, and other measures should be taken to increase palatability. When the average particle size is less than about 400 μm, the disintegrated ODT has a more palatable “creamy” texture in the mouth of the patient.

In some embodiments, the ODT compositions of the present invention also include additional drugs suitable for treating symptoms of allergic rhinitis, the common cold, motion sickness, insomnia, Parkinson's disease, nasal congestion, sinusitis, upper respiratory tract infections, allergies, fever, or additional drugs such as non-opioid analgesics or NSAIDs for treating night time pain and for sleep management. Non-limiting examples of additional drugs include e.g. phenylephrine, pseudoephedrine, hydrocodone, acetaminophen, aspirin, etodolac, diclofenac potassium, ibuprofen, ketoprofen, meloxicam, celecoxib, endomethacin, sulindac, and combinations with diphenhydramine thereof. When present, the additional drugs are in the form of taste-masked drug-containing particles (e.g., crystalline or amorphous drug, granulated drug, or drug-layered beads) analogous to the diphenhydramine-containing particles described herein. Thus, in some embodiments the ODT compositions of the present invention which include additional drugs comprise diphenhydramine-containing particles, one or more different kinds of drug-containing particles, and rapidly dispersing granules. Alternatively, the taste-masked diphenhydramine-containing particles themselves can include a mixture of diphenhydramine and one or more additional drugs. Thus, in some embodiments the ODT compositions of the present invention comprise granules which comprise diphenhydramine and at least one additional drug combined with a pharmaceutically acceptable diluent and/or fillers (as well as rapidly dispersing granules). In other embodiments, the compositions of the present invention comprise drug-layered beads in which a mixture of diphenhydramine and at least one additional drug, in combination with a binder, is layered onto an inert core as disclosed herein. The ODT compositions of the present invention also include combinations of diphenhydramine-containing particles and additional drug-containing particles comprising a mixture of diphenhydramine and at least one additional drug (as well as rapidly dispersing granules). In a particular embodiment, particles of diphenhydramine, hydrocodone, pseudoephedrine, phenylephrine, acetaminophen, aspirin, etodolac, diclofenac, ibuprofen, ketoprofen, meloxicam, celecoxib, endomethacin, and sulinda are individually taste-masked so that therapeutically effective amounts of individual active components are blended together with rapidly-dispersing microgranules and other excipients including a flavor, a sweetener, and a colorant (if needed) and compressed into combination ODT products.

The amount of rapidly dispersing granules or the amount of disintegrant-sugar alcohol/saccharide combination in relation to the taste-masked diphenhydramine-containing particles (and optionally taste-masked additional drug-containing particles of one or more of the drugs selected from the group consisting of diphenhydramine, pseudoephedrine, hydrocodone, phenylephrine, aspirin, etodolac, diclofenac potassium, ibuprofen, ketoprofen, meloxicam, celecoxib, endomethacin, sulindac, etc.) can vary depending upon the desired disintegration rate and the desired organoleptic properties including taste-masking, mouthfeel and aftertaste. The amount of the disintegrant-sugar alcohol/saccharide combination in the compositions of the present invention can range from about 40% to about 95%, including about 40%, about 45%, about 50%, about 55%, about 60%, about 65%, about 70%, about 75%, about 80%, about 85%, about 90%, and about 95%, inclusive of all values, ranges, and subranges therebetween. In one embodiment, the amount of disintegrant-sugar alcohol/saccharide combination is about 60-70% of the total weight of the composition. In another embodiment, the amount of disintegrant-sugar alcohol/saccharide combination is about 65% by weight.

The ODT compositions of the present invention contain a sufficient quantity of taste-masked drug-containing particles to provide a therapeutically effective dose of the component drugs (i.e., diphenhydramine and optional additional drugs). The amount of the drug(s) in individual taste-masked drug-containing particles can be adjusted to provide a therapeutically effective dose of the component drugs.

The amount of the component drugs in the ODT compositions of the present invention can range from about 2% to about 25%, including about 5%, about 10%, about 15%, about 20%, and about 25%, inclusive of all values, ranges, and subranges therebetween.

In one embodiment, and ODT composition of the present invention contains about 6% to about 12% by weight of diphenhydramine hydrochloride. In another embodiment, and ODT composition of the present invention additionally contains about 3% to about 6% by weight of phenylephrine hydrochloride.

In addition to acceptable disintegration and organoleptic properties, commercially acceptable ODT formulations must have hardness and friability suitable for packaging in bottles or in push-through film-backed and/or peel-off paper-backed blister packs for storage, transportation and commercial distribution. Accordingly, in addition to the taste-masked diphenhydramine-containing particles, disintegrant, and sugar alcohol and/or saccharide, the ODT compositions of the present invention can also include other pharmaceutically acceptable ingredients or excipients which aid in forming tablets with acceptable hardness and friability characteristics, promote rapid disintegration, and/or improve the organoleptic properties of the ODT formulations.

Examples of suitable excipients for use in the compositions or dosage forms of the present invention include fillers, diluents, glidants, disintegrants, binders, lubricants etc. Other pharmaceutically acceptable excipients include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include lactose (e.g. spray-dried lactose, α-lactose, β-lactose, Tabletose®, various grades of Pharmatose®, Microtose® or Fast-Floc®), microcrystalline cellulose (e.g. Avicel PH101, Avicel PH102, Ceolus KG-802, Ceolus KG-1000, Prosolv SMCC 50 or SMCC90, various grades of Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A 15C, Methocel A4M), hydroxyethylcellulose, sodium carboxymethylcellulose, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Specific examples of diluents include e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of glidants and lubricants include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients include e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Non-limiting examples of flavoring agents include e.g. cherry, orange, or other acceptable fruit flavors, or mixtures of cherry, orange, and other acceptable fruit flavors, at up to, for instance, about 3% based on the tablet weight. In addition, the compositions of the present invention is can also include one or more sweeteners such as aspartame, sucralose, or other pharmaceutically acceptable sweeteners, or mixtures of such sweeteners, at up to about 2% by weight, based on the tablet weight. Furthermore, the compositions of the present invention can include one or more FD&C colorants at up to, for instance, 0.5% by weight, based on the tablet weight.

Antioxidants include e.g. ascorbic acid, ascorbyl palmitate, butylated hydroxyanisole, butylated hydroxytoluene, hypophosphorous acid, monothioglycerol, potassium metabisulfite, propyl gallate, sodium formaldehyde sulfoxylate, sodium metabisulfite, sodium thiosulfate, sulfur dioxide, tocopherol, tocopherol acetate, tocopherol hemisuccinate, TPGS or other tocopherol derivatives, etc.

For example, the ODT compositions of the present invention can include a synthetic sweetener such as sucralose, a flavoring agent such as a cherry flavor, a tabletting aide such as microcrystalline cellulose, and an additional disintegrant.

When the ODT compositions of the present invention include rapidly dispersing microgranules, the compositions can also include an additional disintegrant. The additional disintegrant can be the same disintegrant used in the rapidly dispersing microgranules, or a different disintegrant. The additional disintegrant may be present in the ODT compositions of the present invention at up to, for instance, about 10% based on the tablet weight.

Specific examples of additional disintegrants include e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc. Specific examples of binders include e.g. acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

In one embodiment, the ODT compositions of the present invention comprise about 15-35% of diphenhydramine crystals, microencapsulated with a taste-masking layer comprising a water-insoluble polymer (e.g., ethylcellulose); about 80-70% of rapidly-dispersing granules (e.g., comprising crospovidone and mannitol); about 5% of additional disintegrant (e.g., crospovidone); about 1% of one or more flavors, and about 0.5%-1% of a sweetener (e.g., sucralose).

The ODT compositions of the present invention comprise a therapeutically effective amount of diphenhydramine coated with a taste-masking layer, e.g. in the form of a tablet further comprising rapidly dispersing granules comprising a disintegrant and a sugar alcohol and/or saccharide. Upon administration, the rapidly dispersing granules of the ODT tablet of the present invention rapidly swells and/or dissolves in the patient's oral cavity, thereby causing disintegration of the ODT tablet into taste-masked, diphenhydramine-containing particles to form a smooth, palatable, easy-to-swallow suspension that can be readily swallowed.

In another embodiment, the ODT compositions of the present invention comprise taste-masked diphenhydramine-containing microparticles, one or more flavoring agents, a sweetener, rapidly-dispersing microgranules, microcrystalline cellulose, an additional disintegrant, and a lubricant such as magnesium stearate, compressed into orally disintegrating tablets. The orally disintegrating tablets formed thereby rapidly disintegrate on contact with saliva in the buccal cavity, and have a pleasant taste (good creamy mouth feel) and provide rapid, substantially-complete release of the diphenhydramine dose in the stomach.

In yet another embodiment, the ODT compositions of the present invention comprise taste-masked drug microparticles (e.g. diphenhydramine-containing microparticles, optionally in combination with one or more of pseudoephedrine-, phenylephrine-, or hydrocodone-containing particles), and optionally flavoring agents, sweeteners, and other pharmaceutically acceptable excipients in a tablet press equipped with an externally lubricating system to pre-lubricate dies and punches, thereby providing an ODT formulation otherwise free of lubricant. The orally disintegrating tablets thus produced typically exhibit sufficient hardness and sufficiently low friability to be suitable for packaging in HDPE bottles and push-through film-backed or peel-off paper backed blister packs using conventional equipment for storage, transportation and commercial distribution. The optional flavoring agents, sweeteners, and other pharmaceutically acceptable excipients, tablet presses, etc., as well as compression conditions include, for example those described in U.S. Published Application Nos. 2007/0196491, 2007/0190145, 2006/0105039, 2006/0105038, 2006/0078614, 2006/0057199, and 2005/0232988, each of which is herein incorporated by reference in its entirety for all purposes.

The rate of disintegration of the ODT compositions of the present invention in the oral cavity of a patient can be on the order of about 60 seconds or less, about 50 seconds or less, about 40 seconds or less, about 30 seconds or less, about 20 seconds or less, or about 10 seconds or less.

The rate of disintegration can also be measured using various in vitro test methods, for example the USP <701> Disintegration Test. When using the USP <701> Disintegration Test, the rates of disintegration of ODT compositions of the present invention are faster than those of conventional, non-ODT immediate release diphenhydramine-containing compositions, for example 60 seconds or less, 30 seconds or less, 20 seconds or less, or 10 seconds or less. (The term “non-ODT immediate release diphenhydramine-containing compositions” refers to conventional tablets or capsules intended to be swallowed and absorb in the gastrointestinal tract, or chewable tablets which require mastication to break apart the tablet structure, and which do not contain extended release or controlled release coatings to delay release of the diphenhydramine).

The dissolution rate of the ODT can be evaluated using the United States Pharmacopoeia Apparatus 2 (paddles @75 rpm in 900 mL of 0.01N HCl buffer). When using the United States Pharmacopoeia Apparatus 2, the rate of dissolution of the drug (e.g., diphenhydramine) is comparable to that of conventional, non-ODT immediate release diphenhydramine-containing compositions, for example about 70%, about 75%, about 80%, about 85%, about 90%, about 95%, or about 100% of the drug (e.g., diphenhydramine) is released in about 30 min.

The ODT compositions of the present invention provide good taste-masking when placed in the mouth until swallowed (e.g., not more than about 10% of the drug dose released in about 3 minutes when tested for dissolution in simulated saliva fluid at pH of about 7.0). An ODT of the present invention will disintegrate in about 30 seconds when evaluated using the USP <701> Disintegration Test, and will typically disintegrate on contact with saliva in the buccal cavity in vivo within about 60 seconds, forming a smooth, easy-to swallow suspension of taste-masked microparticles with an acceptable aftertaste. These taste-masked microparticles will typically provide substantially complete release of the diphenhydramine dose upon entering the stomach (e.g., not less than about 60%, more particularly not less than about 70% of the diphenhydramine dose released in about 30 minutes when tested for dissolution in simulated gastric fluid or 0.01N HCl).

The drug-containing particles (e.g. diphenhydramine-containing particles) of the present invention can be prepared by any suitable method. For example, the drug-containing particles can be prepared by the granulation of drug crystals, one or more disintegrants, and one or more fillers (e.g., sugar alcohol, saccharide and/or microcrystalline cellulose) in a high shear granulator or a fluid-bed granulator using a solution of one or more polymeric binders, and dried in fluid bed equipment or on trays in a conventional oven to produce the diphenhydramine-containing granules.

Alternatively, the drug-containing particles can be prepared by layering a solution of the drug and a polymeric binder, dispersed or dissolved in a pharmaceutically acceptable solvent (e.g., water, alcohols such as ethanol, ketones such as acetone, hydrocarbons such as cyclohexane, and combinations thereof), onto an inert core (e.g., sugar beads, cellulose beads, or silica beads) e.g. in a fluid bed coating apparatus.

In certain embodiments of the present invention, diphenhydramine-containing particles (e.g., diphenhydramine-containing granules, diphenhydramine crystals and/or diphenhydramine-layered beads) are coated with a taste-masking layer by solvent coacervation or microencapsulation by phase separation with a water-insoluble polymer, or a combination of a water-insoluble polymer and a gastrosoluble pore-former, e.g. by the method described in U.S. patent application Ser. No. 11/213,266, which is herein incorporated by reference in its entirety for all purposes. For example, in one embodiment, diphenhydramine HCl is layered on sugar spheres in a fluid-bed granulator and provided with a protective seal-coat (e.g., Opadry Clear). The resulting diphenhydramine HCl layered beads are then taste-masked by microencapsulation (phase separation) in cyclohexane with a water-insoluble polymer (e.g., ethylcellulose) in combination with a gastrosoluble pore-former (e.g., calcium carbonate) to provide taste-masked beads using the method described in U.S. patent application Ser. No. 11/256,653, which is herein incorporated by reference for all purposes. Alternatively, diphenhydramine (or additional drugs) crystals with an average particle size range of about 1-200 μm, more particularly about 50-150 μm, can be coated with a taste-masking layer by either fluid-bed coating or solvent coacervation in accordance with other aspects of the invention. Crystalline diphenhydramine with a mean particle size of about 5-50 μm can also be taste-masked by solvent coacervation as described herein.

If the ODT compositions of the present invention include an additional drug, the additional drug can be present in the form of taste-masked drug-containing particles. The additional drug can be included in the diphenhydramine particles, or in (or on) separate taste masked particles. For example, phenylephrine HCl is granulated with microcrystalline cellulose and a binder, then taste-masked by microencapsulation (phase separation) with ethylcellulose in cyclohexane. The diphenhydramine-containing particles and phenylephrine-containing particles are then mixed with a disintegrant or rapidly dispersing granules, and compressed to form an ODT.

Alternatively, the drug-containing particles (e.g., diphenhydramine-containing granules, diphenhydramine crystals and/or diphenhydramine-layered beads) are taste-masked by fluid-bed coating with a water-insoluble polymer in combination with a gastrosoluble polymer such as Eudragit E100 or EPO (an aminoalkyl methacrylate copolymer) by the method described in U.S. patent application Ser. No. 11/248,596, which is herein incorporated by reference in its entirety for all purposes.

For example, dissolved or suspended drug (e.g. diphenhydramine) in a polymeric binder solution is layered onto inert particles (50-100 mesh or 150-300 μm in diameter) such as sugar spheres or cellulose spheres (e.g., Celphere® CP-203, Cellets® 100 or Cellets® 200) using a fluid-bed coater equipped with a bottom-spray Wurster insert. These drug-layered beads can then be taste-masked by fluid-bed coating or by solvent coacervation as described herein.

In a specific embodiment, a water-insoluble polymer (e.g., ethylcellulose), a phase-inducer (e.g., polyethylene), and diphenhydramine are loaded into a coacervation tank containing cyclohexane. The mixture in the tank is heated to about 80° C. to dissolve the ethylcellulose, and then slowly cooled under controlled conditions thereby causing phase-induced microencapsulation of diphenhydramine-containing particles with the ethylcellulose. Upon reaching ambient temperature, the suspension of microencapsulated diphenhydramine-containing particles are filtered, washed with fresh cyclohexane and dried to reduce residual solvent levels within acceptable limits (e.g., <4,000 ppm), in one embodiment less than 1,000 ppm. The coating weight of the microencapsulated diphenhydramine-containing particles can range from about 5% to about 40% including about 10%, 15%, 20%, and 25%, inclusive of all ranges and subranges therebetween. Examples of such a coacervation process are disclosed in U.S. Pat. Nos. 5,252,337, 5,639,475, 6,139,865 and 6,495,160, each of which is herein incorporated by reference in their entirety for all purposes.

Alternatively, the coacervation solution can comprise a mixture of the water-insoluble polymer (e.g., ethylcellulose) and a water-insoluble or gastrosoluble pore-former (e.g., calcium carbonate). The ratio of water-insoluble polymer to pore-former can range from about 50/50 to 95/05, including about 55/45, about 60/40, about 65/35, about 70/30, about 75/25, about 80/20, about 85/15, and about 90/10, including all ranges and subranges therebetween. The coating weight of the microencapsulated drug particles can range from about 5% to about 30% including about 10%, 15%, 20%, and 25%, inclusive of all ranges and subranges therebetween. In one embodiment, the coacervation step comprises suspending the diphenhydramine-containing particles in a solution of ethylcellulose at about 80° C. in a coacervation tank. During the cooling cycle, the micronized pore-former is introduced into the tank at a temperature of about 58° C., while constantly stirring the suspension to uniformly distribute the pore-former in the microcapsule-membrane, at the forming/hardening phase. Examples of such a coacervation process are disclosed in U.S. patent application Ser. No. 11/213,266.

In one embodiment, the ODT compositions of the present invention are prepared by a method comprising (a) granulating diphenhydramine e.g. with a filler and/or diluent such as a sugar alcohol and/or saccharide, (b) coating the diphenhydramine-containing granules with a taste-masking layer e.g. by fluid bed coating or coacervation, (c) blending the taste-masked diphenhydramine granules with a disintegrant, a sugar alcohol and/or saccharide, and optionally other pharmaceutically acceptable excipients, and (d) compressing the blend into an ODT.

In another embodiment, the ODT compositions of the present invention are prepared by a method comprising (a) granulating diphenhydramine e.g. with a filler and/or diluent such as a sugar alcohol and/or saccharide, (b) coating the diphenhydramine-containing granules with a taste-masking layer e.g. by fluid bed coating or coacervation, (c) granulating a disintegrant and a sugar alcohol and/or saccharide to form rapidly-disintegrating granules, (d) blending the coated diphenhydramine-containing granules and the rapidly-disintegrating granules, and optionally other pharmaceutically acceptable excipients, and (e) compressing the blend into an ODT.

In yet another embodiment, the ODT compositions of the present invention are prepared by a method comprising (a) coating a solution or dispersion of diphenhydramine and a pharmaceutically acceptable binder in a pharmaceutically acceptable solvent onto an inert core and removing the solvent to form a diphenhydramine-layered bead, (b) coating the diphenhydramine-layered beads with a taste-masking layer e.g. by fluid bed coating or coacervation, (c) blending the taste-masked diphenhydramine-layered beads with a disintegrant, a sugar alcohol and/or saccharide, and optionally other pharmaceutically acceptable excipients, and (d) compressing the blend into an ODT.

In still another embodiment, the ODT compositions of the present invention are prepared by a method comprising (a) coating a solution or dispersion of diphenhydramine and a pharmaceutically acceptable binder in a pharmaceutically acceptable solvent onto an inert core and removing the solvent to form a diphenhydramine-layered bead, (b) coating the diphenhydramine-layered beads with a taste-masking layer e.g. by fluid bed coating or coacervation, (c) granulating a disintegrant and a sugar alcohol and/or saccharide to form rapidly-disintegrating granules, (d) blending the taste-masked diphenhydramine-layered beads and the rapidly-disintegrating granules, and optionally other pharmaceutically acceptable excipients, and (e) compressing the blend into an ODT.

In still yet another embodiment, the ODT compositions of the present invention are prepared by a method comprising (a) granulating diphenhydramine with a disintegrant and a sugar alcohol and/or saccharide, (b) coating the diphenhydramine-containing granules with a taste-masking layer e.g. by fluid bed coating or coacervation, (c) optionally blending the taste-masked diphenhydramine granules with other pharmaceutically acceptable excipients, and (d) compressing the blend into an ODT.

In a particular embodiment, the ODT compositions of the present invention are prepared by (a) preparing diphenhydramine-containing particles (e.g., by granulating diphenhydramine crystalline material having an average particle size of about 5-50 μm and one or more diluents/fillers such as lactose, mannitol, microcrystalline cellulose and mixtures thereof, with a polymeric binder in a high-shear granulator or a fluid-bed coater, or diphenhydramine-layered beads by dissolving the diphenhydramine in a polymer binder solution and spraying the diphenhydramine solution onto inert spheres (e.g., sugar spheres or cellulose spheres) in a fluid bed coater and applying a protective seal-coat); (b) taste-masking the diphenhydramine-containing particles by microencapsulation (i.e. coacervation) or fluid bed coating with ethylcellulose alone or in combination with a gastrosoluble calcium carbonate or by fluid bed coating with ethylcellulose and Eudragit E100; (c) granulating one or more sugar alcohols and/or saccharides, each having an average particle diameter of not more than about 30 μm, with a disintegrant such as crospovidone, using water or an alcohol-water mixture in a conventional granulator, and drying the granulate in fluid-bed equipment or a conventional oven to produce rapidly-dispersing microgranules with an average particle size of not more than about 400 μm; (d) blending the taste-masked drug microparticles of step (b) with one or more flavoring agents, a sweetener, microcrystalline cellulose, additional disintegrant, and the rapidly-dispersing microgranules of step (c); and (e) compressing the blend of step (d) into tablets using e.g. a conventional rotary tablet press equipped with an external lubrication system to pre-lubricate the dies and punches.

The rapidly dispersing granules of the present invention can be prepared by any suitable method. For example, the rapidly dispersing granules can be prepared by granulation of one or more disintegrants and one or more sugar alcohols and/or saccharides in a high shear granulator, and dried in fluid bed equipment or on trays in a conventional oven to produce the rapidly dispersing granules, e.g. in the form of rapidly-dispersing microgranules. Rapidly-dispersing microgranules can also be produced by the method described in U.S. patent application Ser. No. 10/827,106, which is herein incorporated by reference in its entirety for all purposes.

In a particular embodiment, the ODT compositions of the present invention are prepared by blending (a) diphenhydramine-containing particles (e.g., diphenhydramine-containing granules, diphenhydramine crystals and/or diphenhydramine-layered beads) taste-masked by any of the methods described in U.S. patent application Ser. Nos. 10/827,106; 11/213,266; 11/248,596; 11/256,653, each of which is herein incorporated by reference in its entirety; (b) rapidly dispersing microgranules are prepared by the method described in the above listed U.S. patent application Ser. No. 10/827,106, and (c) blending the diphenhydramine-containing particles, rapidly dispersing granules, and other pharmaceutically acceptable ingredients such as a flavor, a sweetener, a colorant, an additional disintegrant, and a compression aide such as microcrystalline cellulose, and (d) compressing the mixture into an ODT using a rotary tablet press equipped with an external lubrication system to lubricate die and punch surfaces prior to compression.

### Example 1

### 1A Drug-Layered Beads

Drug Layering Solution: A grounded stainless steel tank equipped with a propeller mixer was filled with 300 kg of Acetone NF. Purified Water USP (93.3 kg) was slowly added to the tank while stirring the tank at approximately 850 rpm±25 rpm. Diphenhydramine hydrochloride (76.5 kg) was slowly added into the tank to dissolve while stirring. Hydroxypropylcellulose (Klucel LF; 8.42 kg) was slowly added into a separate stainless steel tank containing 86.4 kg of acetone and 9.6 kg of water to dissolve.

Drug Layering Method: 60-80 mesh sugar spheres (215 kg) were charged into a preheated Glatt GPCG 120 fluid-bed coater equipped with a bottom spray Wurster insert (see Table 2 for equipment and process parameters). The batch recipe proceeded automatically with the drug layering step at 300 g/min and increase flow rates and inlet temperatures accordingly. Processing parameters were recorded approximately every 30 minutes (minimum). The product was periodically inspected through the sample port to ensure that aggregation does not occur during spraying. Once the coating solution was sprayed onto the sugar spheres, a seal coating was applied at a spray rate of 300 g/min for a 2% weight gain. Following the completion of the seal coating, the beads were dried in the Glatt unit to drive off residual acetone. The diphenhydramine-layered beads thus produced were sieved through #32 and #80 mesh screens into a clean, labeled 30-gallon fiber drums, double-lined with polyethylene bags. Over and under sized beads were discarded.

TABLE 1
Drug Layering of Diphenhydramine HCl

| % Per | | Actual Quantity | |
|---|---|---|---|
| Batch (w/w) | Ingredients | Per Batch | |
| — | Acetone NF* | 300.0 | kg* |
| — | Purified Water USP* | 93.3 | kg* |
| 25.00 | Diphenhydramine Hydrochloride | 76.5 | kg |
| | USP | | |
| 2.75 | Hydroxypropyl Cellulose NF | 8.42 | kg |
| | (Klucel LF) | | |
| 70.25 | Sugar Spheres NF (60-80 mesh) | 215.0 | kg |
| — | Acetone NF* | 86.4 | kg* |
| — | Purified Water USP* | 9.6 | kg* |
| 2.00 | Hydroxypropyl Cellulose NF | 6.12 | kg |
| | (Klucel LF) | | |

*→ Removed during processing

TABLE 2
Equipment and Processing Parameters

| | |
|---|---|
| Product Bowl | BN-2201 (32″ Wurster) |
| Number of Partitions | 1 (23.75 inches long) |
| Air Distribution Plates | Inner: G 1-122-00017-3 |
| | Outer: C 1-12200015-4 |
| Product Support Screen | 100 mesh Screen |
| Partition Height from | Target: 50 mm |
| Distribution Plate | (Range: 45-55 mm) |
| Nozzle Tip Port Size | 1.5 mm |
| Nozzle Cap Height | Flush with nozzle tip |
| Pump Inlet Tubing Size | 1″ (I.D.) tubing |
| Spray Nozzle Tubing | ¼″ (I.D.); |
| | 7/16″ (O.D.) tubing |
| Dedicated Filter Bag (50 μm) | DPH Bag-01 |
| Pump Heads | Masterflex ® L/S ® Easy-load ® |
| (6 in a dual configuration) | II |
| HS Collar | Yes |
| Drug Layering | |
| Process Air Temperature (° C.) | 70 (ramps down from 80° C. Preheat) |
| | (Range: 50-105) |
| Process Air Volume (cfm) | 1500 (Range: 1200-3000) |
| Spray Rate (g/min) | 1500 (ramps up) (Range: 300-2000) |
| Product Temperature (° C.) | 49-51 (increases gradually) |
| | (Range: 28-60) |

### 1B DPH Microencapsulation (Taste-Masking)

Each tank of a twin tank 500-gallon coacervation system was charged with 415 gallons of cyclohexane, 61.5 kg of diphenhydramine hydrochloride-layered beads (prepared as described in 1A, above), 20.5-25.1 kg of ethylcellulose, and 10-25 kg of polyethylene while stirring at 75±5 rpm. The system was subjected to a computer controlled “heat and hold” cycle whereby the contents of the tanks were heated to about 80° C. to completely dissolve the ethylcellulose, and thereafter to a “filter and fluid-bed dry” routine whereby the contents of the tank were cooled to about 30° C. As the temperature fell below about 65° C., the ethylcellulose which is no longer soluble in cyclohexane started precipitating out (assisted by the phase inducer, polyethylene), thereby coating individual diphenhydramine particles to provide taste-masking. Upon cooling to ambient temperature, the microencapsulated diphenhydramine hydrochloride-layered beads thus formed were vacuum filtered, rinsed with fresh cyclohexane and vacuum dried in the fluid bed equipment to achieve a pre-determined residual solvent level. The dried microencapsulated diphenhydramine hydrochloride-layered beads were sieved through a 40 mesh sieve using a Kason siever and discharged into fiber drums double-lined with polyethylene bags. The microencapsulated diphenhydramine hydrochloride-layered beads thus obtained had an assay of approximately 18.4-19.4% diphenhydramine hydrochloride, exhibited a particle size of not more than 10% retained on 20 mesh sieve and not more than 10% passing through 80 mesh sieve, and a mean dissolution of about 11-22% of the total diphenhydramine dose in 5 minutes and about 62-70% of the total diphenhydramine dose in 45 minutes, when dissolution tested in water at 80±5 rpm.

TABLE 3
Microencapsulation Details

| | | Quantity | | Quantity | | | |
|---|---|---|---|---|---|---|---|
| Item # | Ingredient Name | (TK 1201-A) | | (TK 1201-B) | | Total Quantity | |
| 1 | Diphenhydramine HCl | 61.5 | Kg | 61.5 | Kg | 123.0 | Kg |
| | Intermediate Beads | | | | | | |
| 2 | Ethylcellulose NF | 20.5-25.1 | Kg | 20.5-25.1 | Kg | 41.0-50.2 | Kg |
| 3 | Cyclohexane | 415 | Gal | 415 | Gal | 830 | Gal |
| 4 | Polyethylene* | 10.0-25.0 | Kg | 10.0-25.0 | Kg | 20.0-50.0 | Kg |

### 1C Rapidly Dispersing Microgranules

The rapidly dispersing microgranules may comprise a sugar alcohol such as mannitol and/or a saccharide such as lactose and a super disintegrant such as Crospovidone. The sugar alcohol and/or saccharide and disintegrant will typically be present in the rapidly dispersing microgranules at a ratio of from about 99:1 to about 90:10 (sugar alcohol and/or saccharide:disintegrant). For example, D-mannitol, a sugar alcohol with an average particle size of about 15 μm and Crospovidone XL-10, a disintegrant, were mixed at a ratio of about 95/5 in a high shear granulator using purified water as the granulating fluid and dried by spreading on trays in a heated convection oven, or following the procedure disclosed in the co-pending U.S. patent application Ser. No. 10/827,106 (published as US Patent Application Publication No. U.S. 2005/0232988 on Oct. 20, 2005, the contents of which are hereby incorporated by reference for all purposes). D-mannitol with an average particle size of approximately 20 μm or less (e.g., Pearlitol 25 from Roquette, France) are blended with 8 kg of cross-linked povidone (e.g., Crospovidone XL-10 from ISP) in a high shear granulator (GNX 600 from Vector) and granulated with purified water and wet-milled using Comil from Quadro and tray-dried to obtain a loss on drying (LOD) of less than about 1%. The dried granules are sieved, and oversized material is milled to produce rapidly dispersing microgranules with an average particle size in the range of approximately 175-300 μm.

### 1D DPH ODT Formulation Optimization

The excipients, cherry flavor, sucralose, and crospovidone were pre-blended with microcrystalline cellulose in a small V-blender and milled through a Comil mill with additional microcrystalline cellulose until a homogeneous mixture was obtained. This blend was further blended with microencapsulated DPH (e.g., prepared as described in 1B, above) and rapidly-dispersing granules (e.g., prepared as described above in 1C; see Table 4, below, for a similar composition) for approximately 10 minutes in another V-blender to provide a 12.5 mg diphenhydramine hydrochloride (12.5 mg as DPH salt composition with a tablet weight of 450 mg. Similarly two other blends of 12.5 mg DPH ODT formulations with a tablet weight of 400 mg or 500 mg were also prepared.

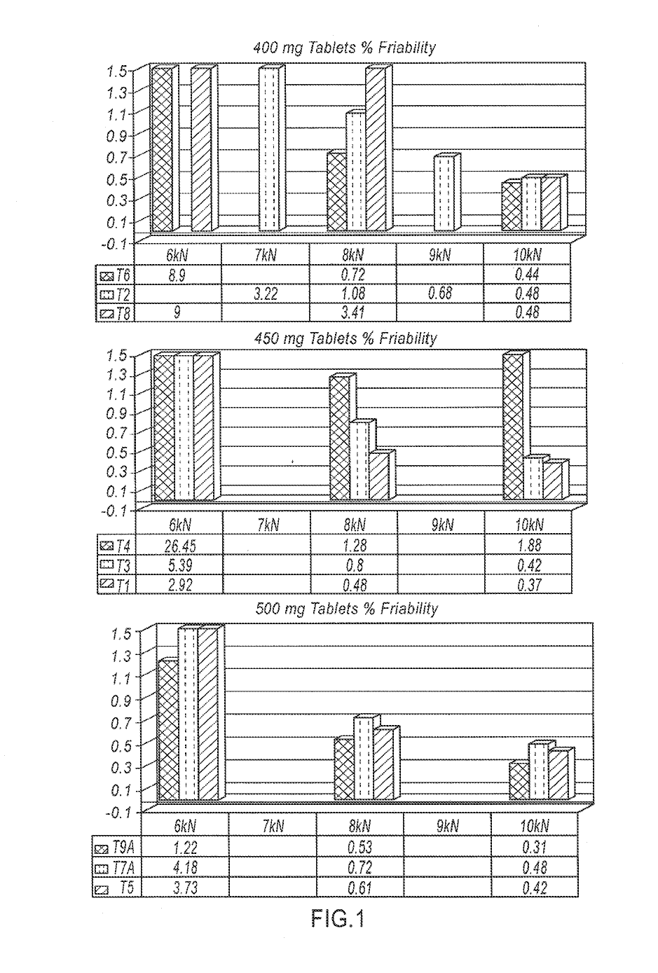

A Hata production tablet press equipped with 11 mm, flat face radius edge tooling and a Matsui ExLube system was adjusted to provide tablets with a friability of less than 1% and a hardness of about 30 N by varying the compression forces from about 6 kN to 10 kN. The ODT compositions (e.g., contents of filler, disintegrant, sweetener, flavor, and rapidly dispersing microgranules), lubricant spray conditions, and tabletting parameters (compression force, fill depth, tablet weight, turret speed, hardness, friability (target: <0.6%)) were varied to demonstrate robustness of the formulations. FIG. 1 shows the variation of tablet friability as a function of compression force at three tablet weights, 400, 450 and 500 mg.

TABLE 4
Composition of DPH ODT 12.5 mg

| Item | Quantity/ | | |
|---|---|---|---|
| No. | Tablet (mg) | % | Ingredient |
| 1 | 66.98 | 14.88 | Microencapsulated Diphenhydramine |
| | | | HCl Beads |
| 2 | 256.05 | 56.90 | Rapidly Dispersing Granules |
| 3 | 90.00 | 20.00 | Microcrystalline Cellulose NF, |
| | | | (Avicel PH 101) |
| 4 | 22.50 | 5.00 | Crospovidone XL-10 NF |
| 5 | 1.17 | 0.26 | D & C Red # 7, Calcium Lake |
| | | | (17%-25%) |
| 6 | 7.20 | 1.60 | Artificial Cherry Flavor |
| | | | Powder # SN 340396 |
| 7 | 1.575 | 0.35 | Sucralose Micro Powder NF, (Splenda) |
| 8 | 4.50 | 1.00 | Citric Acid USP Fine Powder |
| 9 | Trace | Trace | Magnesium Stearate NF, Vegetable |
| | amount | amount | |
| | 450.00 | 100.00 | |

### 1E DPH ODT Blending

A 10.0 cu-ft. V-blender was charged with the excipients in the following order: ˜25.0 kg of microcrystalline cellulose (Avicel® PH 101), 30.0 kg of Crospovidone XL-10, 1.56 kg D&C Red #7, 9.60 kg of Artificial Chemy #13571401 (a flavor powder), 2.10 kg of sucralose, 6.00 kg of citric acid (fine powder), and ˜25.0 kg of microcrystalline cellulose (Avicel® PH 101). The contents were mixed for 10 minutes at 17.5±0.5 rpm. About 35.0 kg of microcrystalline cellulose, the above blended excipients, and an additional ˜35.0 kg of microcrystalline cellulose were sieved using a Comil mill operated at about 60 Hz.

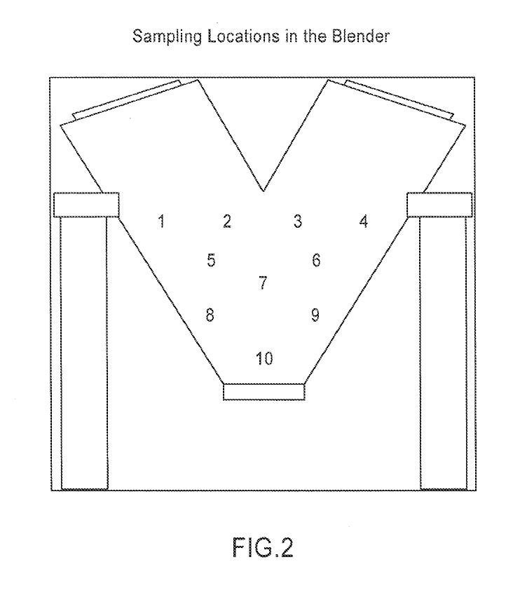

A 50 cu-ft V-blender was charged with ingredients in the following order: ˜half of the rapidly-dispersing granules prepared as described in 1C, all of the microencapsulated DPH (prepared as disclosed in Example 1B), and all of excipients blended in the Comil mill, above, and the remaining rapidly-dispersing granules and blended @6±0.5 rpm for 30±1 minutes to achieve blend homogeneity, and discharged into 30 gallon drums with double-lined polyethylene bags. In order to demonstrate the robustness of the manufacturing processes, several studies based on “design of experiment (DOE)” varying critical operating parameters, such as the method of adding ingredients into the V-blender or the tablet press hopper, time of blending, turret speed, compression force, etc., were carried out. In one such experiment, the compositions were prepared in two different blenders, a 10 cu-ft blender (batch size: 150 kg) and a 50 cu-ft blender (batch size: 600 kg) for varying time periods in order to demonstrate blend homogeneity (see FIG. 2 for sampling locations). The results are shown in Table 5.

TABLE 5
Blend homogeneity data for DPH ODT blends

| 10 cu/ft Blender | | | | | 50 cu/ft Blender |
|---|---|---|---|---|---|
| Blender RPM = 17.5 | | | | | Blender RPM = 6.0 |
| Batch # | | | | Batch # | Batch # |
| 1165-CK-015 | | | | 1165-CK-017 | 1165-CK-018 |
| Sample/ | 6 min. | 9 min. | 11 min. | 11 min. | 30 min. |
| Location | (%) | (%) | (%) | (%) | (%) |
| 1A | 97.9 | 98.5 | 106.2 | 97.1 | 100.2 |
| 2A | 97.4 | 97.0 | 100.4 | 96.4 | 99.2 |
| 3A | 97.9 | 96.4 | 102.3 | 95.9 | 103.2 |
| 4A | 94.9 | 100.5 | 98.0 | 94.0 | 102.1 |
| 5A | 101.8 | 103.1 | 97.7 | 97.5 | 102.5 |
| 6A | 98.5 | 101.5 | 98.1 | 85.9 | 104.5 |
| 7A | 99.8 | 102.1 | 95.8 | 95.5 | 102.9 |
| 8A | 97.7 | 103.4 | 100.7 | 101.7 | 101.1 |
| 9A | 96.4 | 100.5 | 96.9 | 98.1 | 106.7 |
| 10A | 98.1 | 100.5 | 98.6 | 103.1 | 105.8 |
| Avg. | 98.0 | 100.4 | 99.5 | 96.5 | 102.8 |
| % RSD | 1.88 | 2.38 | 3.06 | 4.82 | 2.3 |

### 1F DPH ODT, 12.5 mg

A Hata production tablet press equipped with a vacuum transfer system (tooling description: 11 mm, round flat face radius edge tooling, tablet de-duster, a metal detector, and a Matsui ExLube system was adjusted to provide tablets with a friability of less than 1% and a hardness of about 30 N by varying the compression forces from about 6 kN to 10 kN. Magnesium stearate was used as a processing aid, i.e., to externally lubricate the punch and die surfaces, and hence was present in trace amounts on the tablets. The weight range for the tablets was typically ±5% of the target tablet weight. The ExLube system was started to ensure that the lubricant was spraying properly when the tablet press was running. The tabletting parameters such as fill depth (mm), pre-compression position (mm or kN) and main compression position (mm or kN) were adjusted on the press in order to produce 12.5 mg DPH tablets that meet the specifications listed below as an example.

Operation Parameters

| | Target | Range |
|---|---|---|
| Hata Tablet Press | | |
| Turntable Speed (RPM) | 25 | 15-35 |
| Fill Depth (mm) | 8.45 | 8.10-9.10 |
| Main Position (mm) | 2.53 | 2.20-2.85 |
| Pre Position (mm) | 3.07 | 2.70-3.40 |
| Scale on the feed shoe | 2.0 | 2-8 |
| Tablet Parameters | | |
| Weight (mg) | 450 | 437-464 |
| Thickness (mm) | 4.80 | 4.40-5.10 |
| Hardness (N) | 33.0 | 23.0-43.0 |
| Friability (%) | NMT 0.6% | NMT 1.0% |

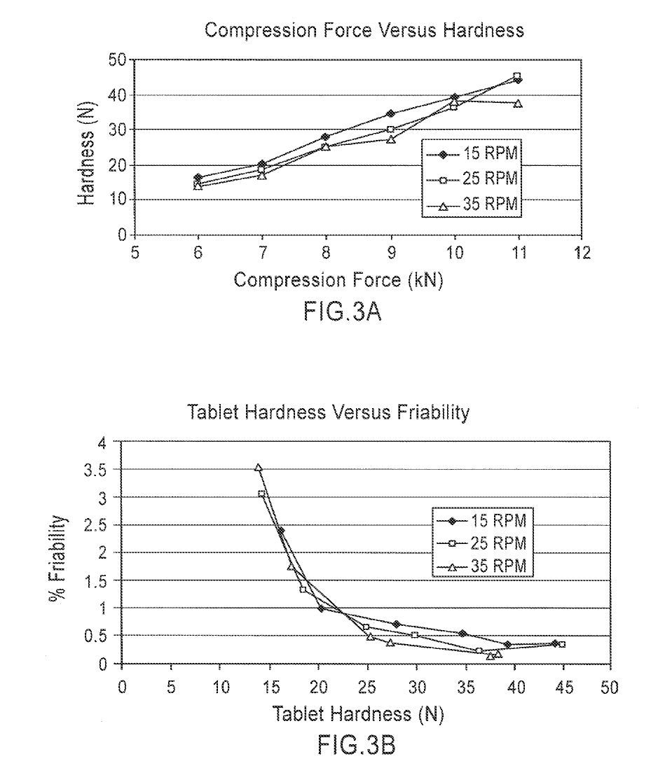

Following set-up, the press was run in ‘Automatic Mode’ until the tableting run was completed. During the run, the tablets were sampled periodically to ensure that they met the specifications listed above. Some of the DOE (design of experiment) blends were compressed into tablets at different compression forces. The tabletting properties are shown in FIGS. 3A and 3B. The dissolution profiles in 900 mL water (paddles at 75 rpm) for a single ODT batch compressed at two compression forces are shown in the table below.

| | |
|---|---|
| Tablet | % Dissolved at |

| Hardness | 5 min | 15 min. | 30 min. | 45 min. | 60 min. |
|---|---|---|---|---|---|
| 19N | 37 | 66 | 88 | 101 | 109 |
| 39N | 37 | 65 | 86 | 96 | 101 |

### 1G DPH ODT, 25 and 50 mg

OUT tablets containing 25 and 50 mg doses of diphenhydramine hydrochloride (see Tables 6 and 7 for compositions) weighing approximately 650 and 1300 mg, respectively, were prepared following the procedure described above. Following set-up, the press was run in ‘Automatic Mode’ until completion. During the run, tablets were sampled periodically to ensure that they would meet the specifications listed above.

TABLE 6
Composition of DPH ODT 25 mg

| Item | Quantity/ | | |
|---|---|---|---|
| No. | Tablet (mg) | % | Ingredient |
| 1 | 132.28 | 20.35 | Microencapsulated Diphenhydramine |
| | | | HCl Beads |
| 2 | 334.36 | 51.44 | Rapidly Dispersing Granules |
| 3 | 130.00 | 20.00 | Microcrystalline Cellulose NF, |
| | | | (Avicel PH 101) |
| 4 | 32.50 | 5.00 | Crospovidone XL-10 NF |
| 5 | 1.69 | 0.26 | D & C Red # 7, Calcium Lake |
| | | | (17%-25%) |
| 6 | 10.40 | 1.60 | Artificial Cherry Flavor |
| | | | Powder # SN 340396 |
| 7 | 2.28 | 0.35 | Sucralose Micro Powder NF, (Splenda) |
| 8 | 6.50 | 1.00 | Citric Acid USP Fine Powder |
| 9 | Trace | Trace | Magnesium Stearate NF, Vegetable |
| | amount | amount | |
| | 650.01 | 100.00 | |

TABLE 7
Composition of DPH ODT 50 mg

| Item | Quantity/ | | |
|---|---|---|---|
| No. | Tablet (mg) | % | Ingredient |
| 1 | 264.55 | 20.35 | Microencapsulated Diphenhydramine |
| | | | HCl Beads |
| 2 | 671.32 | 51.64 | Rapidly Dispersing Granules |
| 3 | 260.00 | 20.00 | Microcrystalline Cellulose NF, |
| | | | (Avicel PH 101) |
| 4 | 65.00 | 5.00 | Crospovidone XL-10 NF |
| 5 | 3.38 | 0.26 | D & C Red # 7, Calcium Lake |
| | | | (17%-25%) |
| 6 | 18.20 | 1.40 | Artificial Cherry Flavor |
| | | | Powder # SN 340396 |
| 7 | 4.55 | 0.35 | Sucralose Micro Powder NF, (Splenda) |
| 8 | 13.00 | 1.00 | Citric Acid USP Fine Powder |
| 9 | Trace | Trace | Magnesium Stearate NF, Vegetable |
| | amount | amount | |
| | 1300.0 | 100.00 | |

### Example 2

### 2A Hydrocodone Drug-Layered Beads

Hydroxypropylcellulose (Nisso HPC-L-FP; 8.1 g) was slowly added to a mixture of 1453 g of acetone and 782 g of water in a stainless steel mixer, with agitation, until dissolved. Hydrocodone bitartrate (“HB”, 81.1 g) was slowly added into the hydroxypropylcellulose solution until dissolved. A Glatt GPCG 3 fluid bed granulator/particle coater equipped with a 7″ bottom spray Wurster insert was charged with 1500 g of 60-80 mesh sugar spheres, and layered with a hydrocodone solution using a bottom air distribution ‘C’ plate, an atomization air pressure of 2.5 bar, and a nozzle port size of 1.0 mm. A 2% by weight seal coat of hydroxypropylcellulose (HPC) was applied on the hydrocodone-layered beads, which were then dried in the Glatt unit to minimize residual solvent.

Hydrocodone bitartrate drug layered beads coated with a protective seal coat are similarly prepared for a drug load of 8.77%.

### 2B Taste-Masked Hydrocodone Bitartrate (30% Coating)

A 4 L solvent coacervation tank is charged with 2 kg of cyclohexane and further charged with 140 g of hydrocodone-layered beads, prepared as described in 2B, above, 60 g of ethylcellulose (Ethocel Premium 100 cps from Dow Chemicals) and 40 g of Polyethylene (Epolene C-10 wax). The tank is heated to about 80° C. to dissolve the ethylcellulose. The contents of the tank are cooled to below 30° C. while stirring at 300 RPM and the resulting ethylcellulose encapsulated hydrocodone-layered beads are filtered, then washed with fresh cyclohexane to remove polyethylene, and dried overnight in the hood.

### Example 3

### 3A Phenylephrine HCl Microgranules (drug load: 15%)

Povidone (0.35 kg) was slowly added to 40.3 kg of water in a stainless steel tank until dissolved, while stirring at 750±25 rpm. Then phenylephrine HCl (6.75 kg) was slowly added into the povidone solution until dissolved. A Fluid Air FA0300 fluid bed granulator equipped with a BN-1401 product bowl, 100 mesh product support screen, an assembly of three nozzles with a nozzle tip of 0.085″ and a 2-head peristaltic pump, was charged with 37.9 kg of microcrystalline cellulose (Avicel PH 102), and granulated by spraying the drug solution at the following coating conditions: inlet temperature target: 75° C.; fluidization air volume target: 1000 cfm; spray pump setting: 18% and product temperature target: 40° C. After a spraying rinse volume (water), the granules were dried for 15 minutes.

### 3B Phenylephrine HCl Taste-Masking (30% Coating)

A 200 gallon coacervation tank was charged with 112 gallons of Cyclohexane and Ethocel Premium 100 cps (16.3 kg) and Epolene (2.6 kg), while stirring at 60+5 rpm. Phenylephrine HCl (PE) microgranules prepared as described above in 3A were then added to the coacervation tank. The contents of the tank were then subjected to a computer controlled “heat and hold” cycle, and thereafter to a “controlled cooling” cycle, thereby providing ethylcellulose encapsulated (taste-masked) phenylephrine-containing microgranules. The taste-masked phenylephrine granules were then recovered by filtration and dried in the fluid bed drier.

### 3C ODTs Comprising 12.5-mg DPH/5-mg PE

A 2.0 cu-ft. V-blender (see Table 8 for compositions) was charged with excipients in the following order: 4.75 kg of microcrystalline cellulose, (Avicel PH 101), 4.75 kg of Crospovidone XL-10, 0.180 kg D&C Red #27, Alum, 0.057 kg FD&C Blue #1, 1.045 kg of Grape Permaseal Art. #184557 (a flavor), 0.333 kg of Sucralose, 1.90 kg of citric acid (fine powder), and another 4.75 kg of microcrystalline cellulose (Avicel PH 101). These excipients were mixed for 10 minutes. An additional 4.75 kg of microcrystalline cellulose (Avicel PH 101), the above blended excipients, and a final 4.75 kg aliquot of microcrystalline cellulose (Avicel PH 101) were sieved using a Comil mill and then discharged into double-lined polyethylene bags.

The 10 cu-ft V-blender was charged with the ingredients in the following order: about half (−21.77 kg) of the rapidly-dispersing granules prepared as described in 1C, 14.136 kg of taste-masked DPH prepared as disclosed in 1B, 10.05 kg of taste-masked PE granules prepared as disclosed in 3B, all of the Comil blended excipients (above), and the remaining (21.778 kg) of the rapidly-dispersing granules. The ingredients were blended at 17.5±5% rpm and discharged into 30 gallon drums with double-lined polyethylene bags (batch size: 95 kg or approximately 211,000 tablets).

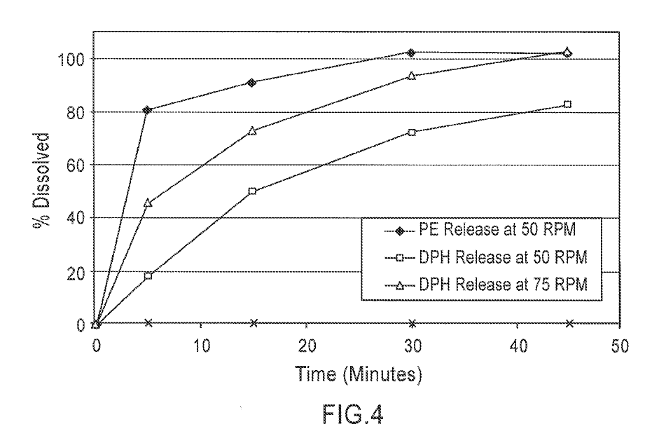

The Hata production press was set up in a manner similar to that described above in 1F for ODT tablets comprising 12.5 mg diphenhydramine HCl and 5 mg phenylephrine HCl, both appropriately taste-masked weighing approximately 450 mg. The press was run in “Automatic Mode” until completion. During the run, tablets were sampled periodically to ensure that they would meet the specifications listed above. The dissolution profiles for scale-up ODT tablet batches are shown in FIG. 4.

TABLE 8
Compositions of ODTs (12.5-mg DPH/5-mg
PE) or (25-mg DPH/5-mg PE/5-mg HB)

| Item | DPH/ | DPH/ | |
|---|---|---|---|
| No. | PE (%) | PE/HB (%) | Ingredient |
| 1 | 14.88 | 13.40 | Microencapsulated Diphenhydramine |
| | | | HCl Beads |
| 2 | 10.58 | 4.76 | Microencapsulated PE HCl Granules |
| 3 | | 8.14 | Microencapsulated Hydroodone |
| | | | Bitartrate Beads from 2B |
| 4 | 45.84 | 49.43 | Rapidly Dispersing Granules |
| 5 | 20.00 | 16.00 | Microcrystalline Cellulose NF, |
| | | | (Avicel PH 101) |
| 6 | 5.00 | 5.00 | Crospovidone XL-10 NF |
| 7 | 0.19 | 0.26 | F D & C Red # 27, Alum Lake |
| 8 | 0.06 | 0.06 | F D &C Blue # 1, Alum Lake |
| 9 | 1.10 | 1.60 | Grape Permaseal Art. #184557 |
| 10 | 0.35 | 0.35 | Sucralose Micro Powder NF, (Splenda) |
| 11 | 2.00 | 1.00 | Citric Acid USP Fine Powder |
| 12 | Trace | Trace | Magnesium Stearate NF, Vegetable |
| | amount | amount | |
| | 100.00 | 100.00 | |

### 3D ODTs Comprising 25-ma DPH/5-mg PE/5-mg HB

A V-blender is charged with excipients (see Table 8 for compositions) in the following order: about 4 parts microcrystalline cellulose, (Avicel PH 101), 5 parts of Crospovidone XL-10, 0.26 part D&C Red #27, 0.06 part of FD&C Blue #1, 1.1 part of Grape Permaseal Art. #184557 (a flavor), 0.35 part of Sucralose, 1 part of citric acid (fine powder), and another 4 parts of microcrystalline cellulose (Avicel PH 101). These excipients are mixed for 10 minutes. An additional 4 parts of microcrystalline cellulose (Avicel PH 101), the above blended excipients, and a final 4 parts aliquot of microcrystalline cellulose (Avicel PH 101) are sieved using a Comil mill and then discharged into double-lined polyethylene bags.

Another V-blender of appropriate capacity, is charged with the ingredients in the following order about half (˜24.9 parts) of the rapidly-dispersing granules prepared as described in 1C, 13.4 parts of the taste-masked DPH prepared in 1B, 8.14 parts of HB prepared in 2B and 4.76 parts of PE prepared in 3B, all of the Comil blended excipients (above) and the remaining (49.43-24.9=24.53 parts) of rapidly-dispersing granules. The ingredients are blended at 17.5±5% rpm and compressed into 25-mg DPH/5-mg PE/5-mg HB weighing about 1000 mg on the Hata tablet press using the ExLube system.

### Example 4

### 4A Taste-masked Pseudoephedrine HCl (40% coating)

Each tank of a twin tank 500-gallon coacervation system was charged with 415 gallons of cyclohexane, 55.5 kg of pseudoephedrine hydrochloride (PSE), 37 kg of ethylcellulose and 24.5 kg of polyethylene while stirring at 57±1 rpm. The contents of the tanks were subjected to a computer controlled “heat and hold” cycle, and thereafter to a “filter and fluid-bed dry” routine. Upon drying, the ethylcellulose encapsulated pseudoephedrine hydrochloride was sieved using a Kason siever through 40 mesh sieve and discharged into 41 gallon drums double-lined with polyethylene bags.

### 4B ODTs Comprising 25-mg DPH/5-mg HB/75-mg PSE

A V-blender is charged with excipients (see Table 9 for details) in the following order: ˜3 parts of Microcrystalline cellulose (Avicel PH 101), 1 part of Citric acid (fine powder), 1 part of Chemy flavor, 0.5 part of Sucralose, 0.35 part of F D & C Red and ˜3 parts microcrystal line cellulose. The excipients are mixed for about 10 minutes. About 3 parts of microcrystalline cellulose, the above blended excipients, and the remaining ˜3 parts of microcrystalline cellulose are then sieved using a Comil mill and discharged into double-lined polyethylene bags. Another V-blender of appropriate capacity is charged with ingredients in the following order: ˜half (˜19.86 parts) of the rapidly-dispersing granules, 13.4 parts of taste-masked DPH, 8.14 parts of HB and 18.75 parts of PSE, all of Comil material and the remaining (˜20 parts) rapidly-dispersing granules and blended for 15 minutes and discharged into fiber drums with double-lined polyethylene bags. Following successful set-up for approximately 1000 mg ODT tablets comprising 25-mg diphenhydramine HCl, 5-mg hydrocodone bitartrate, and 75-mg pseudoephedrine HCl, the press is run in “Automatic Mode” until completion. During the run, tablets are sampled periodically to ensure that tablets produced meet the specifications listed above.

TABLE 9
Compositions of ODTs (25-mg DPH/5-mg HB/75-mg
PSE HCl or 25-mg DPH/250-mg Acetaminophen)

| Item | DPH/ | DPH/ | |
|---|---|---|---|
| No. | HB/PSE (%) | APAP (%) | Ingredient |
| 1 | 13.40 | 13.40 | Microencapsulated Diphenhydramine |
| | | | HCl Beads |
| 2 | 18.75 | | Taste-masked PSE HCl from 4B |
| 3 | 8.14 | | Microencapsulated HB Beads |
| 4 | | 28.09 | Taste-masked Acetaminophen (APAP) |
| 5 | 39.86 | 38.66 | Rapidly Dispersing Granules |
| 6 | 12.00 | 12.00 | Microcrystalline Cellulose NF, |
| | | | (Avicel PH 101) |
| 7 | 5.0 | 5.00 | Crospovidone XL-10 NF |
| 8 | 0.35 | 0.35 | FD & C Red # 7, Calcium Lake |
| | | | (17%-25%) |
| 9 | 1.00 | 1.00 | Artificial Cherry Flavor |
| | | | Powder # SN 340396 |
| 10 | 0.50 | 0.50 | Sucralose Micro Powder NF, (Splenda) |
| 11 | 1.00 | 1.00 | Citric Acid USP Fine Powder |
| 12 | Trace | Trace | Magnesium Stearate NF, Vegetable |
| | amount | amount | |
| | 100.00 | 100.00 | |

### Example 5

### 5A Acetaminophen Microcapsules

Microcapsules of acetaminophen were manufactured by suspending acetaminophen (semi-fine grade; 216 kg) in commercial scale equipment (e.g., a 500-gallon system-single tank, 326 gallon cyclohexane) at an agitation speed of 90±2 rpm, a target heating temperature of 80° C. to allow dissolution of ethylcellulose (Ethocel Standard 100 Premium; 24 kg) and polyethylene wax (a phase-inducer; 4.8 kg) and cooling temperature of (NMT 35° C.) to allow consolidation of the coating (e.g., approximately 10% to achieve effective taste-masking). The tank was heated to target temperature of 80° C. (range: 78-85° C.) using a computer recipe to dissolve ethylcellulose with a heat and hold time of not less than 65 min to ensure complete dissolution of the coating material. The batch was cooled from 80° C. to below 35° C. in not less than 45 minutes to insure that the membrane wrapping the drug cores was complete. According to the consolidated process, the microcapsules following vacuum filtration and rinsing with fresh cyclohexane were dried in a fluid bed dryer, where microcapsules drying was achieved by a stepwise heating (e.g., inlet temperature set at 35° C., 45° C., and finally at 95° C.) for a period of (4.0±2.0) hours and subsequent cooling; to achieve a residual cyclohexane level of NMT (not more than) 1000 ppm.

### 5.B ODT Blend Comprising 25-mg DPH/250-mg Acetaminophen

A V-blender is charged with excipients in the following order: ˜3 parts of Microcrystalline cellulose (Avicel PH 101), 1 part of Citric acid (fine powder), 1 part of cherry flavor, 0.5 part of sucralose, 5 parts of crospovidone, and 5 parts of microcrystalline cellulose. The excipients are mixed for about 10 minutes. About 3 parts of microcrystalline cellulose, the above blended excipients, and the remaining 5 parts of microcrystalline cellulose are then sieved using a Comil mill and discharged into double-lined polyethylene bags. Another V-blender of appropriate capacity is charged with ingredients in the following order: ˜half (19.66 parts) of the rapidly-dispersing granules, 13.4 parts of taste-masked DPH and 28.09 parts of taste0masked acetaminophen (APAP), all of Comil material and the remaining (19.00 parts) of the rapidly-dispersing granules and blended for 15 minutes and discharged into fiber drums with double-lined polyethylene bags. Following successful set-up for ODT tablets comprising 25-mg diphenhydramine HCl and 250-mg acetaminophen weighing approximately 1000 mg, the press is run in “Automatic Mode” until completion. During the run, tablets are sampled periodically to ensure that tablets produced meet the specifications listed above.

### In Vitro Disintegration Time/Dissolution Testing

Disintegration times were measured using the USP <701> Disintegration Test procedures. The taste-masking property of the taste-masked microparticles and the orally disintegrating tablets were evaluated by determining the percentage of drug-release when tested for dissolution using USP Apparatus 2 (paddles @75 rpm) in 900 mL of saliva-simulating fluid at a pH of about 6.8-7.0 (a release of not more than about 10% of the dose in about 3 minutes is considered acceptable). In addition, the rapid-release property in the stomach of the taste-masked microparticles and the orally disintegrating tablets were evaluated by determining the percentage of drug dissolved when tested for dissolution using USP Apparatus 2 (paddles @75 rpm) in 900 mL of 0.01N HCl at 37.0±0.5° C. (a release of not less than about 70% of the dose in about 30 minutes is considered acceptable). The potency of the tablets and the percentage of drug dissolved at different time points are determined using a validated HPLC methodology.

It is to be understood that while the invention has been described in conjunction with specific embodiments thereof, that the description above as well as the examples herein are intended to illustrate and not limit the scope of the invention. Any modification within the scope of the invention will be apparent to those skilled in the art to which the invention pertains.

All patents, applications, and other documents cited herein are herein incorporated by reference in their entirety for all purposes.

1-28. (canceled)

29. A composition comprising:
a therapeutically effective amount of diphenhydramine-containing particles coated with a taste-masking layer; and
rapidly dispersing granules comprising at least one disintegrant, and at least one sugar alcohol and/or at least one saccharide;
wherein the taste-masking layer comprises a water-insoluble polymer in combination with a water-soluble or gastrosoluble pore former or a combination thereof.

30. The composition of claim 29, wherein the taste-masking layer comprises a water-insoluble polymer in combination with a water-soluble pore former, and the water-soluble pore former is selected from the group consisting of sucrose, sodium chloride, povidone, and mixtures thereof.

31. The composition of claim 29, wherein the taste-masking layer comprises a water-insoluble polymer in combination with a gastrosoluble pore former, and the gastrosoluble pore former is selected from the group consisting of calcium carbonate, magnesium hydroxide, aminoalkyl methacrylate copolymers, polyvinylacetal diethylaminoacetate, and mixtures thereof.

32. The composition of claim 29, wherein the taste-masking layer has a ratio of water-insoluble polymer to water-soluble or gastrosoluble pore former that ranges from about 90/10 to about 50/50.

33. The composition of claim 29, wherein the water-insoluble taste-masking polymer is ethylcellulose having a viscosity of about 10-100 cps when tested as a 5 weight % solution at ambient temperature.

34. The composition of claim 29, wherein the composition disintegrates within about 30 seconds when tested by the <USP 701> Disintegration Test.

35. The composition of claim 29, wherein the composition releases about 70% or More of the diphenhydramine in 30 minutes when tested for dissolution using United States Pharmacopoeia Apparatus 2 (paddles @75 rpm in 900 mL of 0.01N HCl buffer).

36. The composition of claim 29, further comprising at least one additional drug selected from the group consisting of phenylephedrine, pseudoephedrine, hydrocodone, and combinations thereof.

37. The composition of claim 29, further comprising at least one additional drug selected from the group consisting of acetaminophen, ibuprofen, meloxicam, ketoprofen, aspirin, celecoxib, etodolac, sulindac, endomethacin, diclofenac, and combinations thereof.

38. The composition of claim 36, wherein the additional drug is present in the form of a taste-masked drug-containing particle.

39. The composition of claim 36, wherein the additional drug is included in the diphenhydramine containing particles.

40. The composition of claim 37, wherein the additional drug is present in the form of a taste-masked drug-containing particle.

41. The composition of claim 37, wherein the additional drug is included in the diphenhydramine containing particles.

42. The composition of claim 29, wherein the diphenhydramine-containing particles have an average particle size of about 1-100 μm prior to coating with the taste-masking layer, and the diphenhydramine-containing particles coated with a taste-masking layer have an average particle size of about 400 μm or less.

43. The composition of claim 29, wherein the diphenhydramine-containing particles are drug-layered beads comprising an inert core coated with a diphenhydramine-containing layer.

44. The composition of claim 29, comprising from about 1% to about 30% by weight of diphenhydramine-containing particles coated with a taste-masking layer.

45. The composition of claim 29, wherein the water-insoluble polymer is selected from the group consisting of ethylcellulose, cellulose acetate, cellulose acetate butyrate, polyvinyl acetate, neutral methacrylic ester copolymers, ammonio-methacrylate copolymers and mixtures thereof.

46. The composition of claim 29, wherein the ratio of sugar alcohol and/or saccharide to disintegrant ranges from about 90/10 to about 99/1.

47. The composition of claim 29, wherein the disintegrant is selected from the group consisting of crospovidone, sodium starch glycolate, crosslinked sodium carboxymethyl cellulose, low-substituted hydroxypropylcellulose and mixtures thereof.

48. The composition of claim 29, wherein the sugar alcohol and/or saccharide are selected from the group consisting of mannitol, xylitol, sorbitol, maltitol, and mixtures thereof.

49. A method of preparing the composition of claim 29, comprising:
(a) preparing particles comprising diphenhydramine;
(b) coating the diphenhydramine-containing particles with a taste-masking layer;
(c) mixing the coated diphenhydramine-containing particles of step (b) with rapidly disintegrating granules comprising at least one disintegrant and at least one sugar alcohol and/or at least one saccharide, and optionally other pharmaceutically acceptable ingredients; and
(d) compressing the mixture into tablets.

50. The method of claim 49 further comprising:
(a1) preparing drug-containing particles comprising hydrocodone, pseudoepedrine, acetaminophen, ibuprofen, meloxicam, ketoprofen, aspirin, celecoxib, etodolac, sulindac, endomethacin, or diclofenac; and
(b1) coating said drug-containing particles with a taste-masking layer;
wherein step (c) comprises mixing the coated diphenhydramine-containing particles of step (b), the rapidly disintegrating granules comprising at least one disintegrant and at least one sugar alcohol and/or at least one saccharide, the coated drug-containing particles of step (b1), and optionally other pharmaceutically acceptable ingredients.

51. The method of claim 49, wherein said step (a) comprises dissolving diphenhydramine and a binder in a pharmaceutically acceptable solvent to form a diphenhydramine-layering solution;
coating the diphenhydramine-layering solution onto an inert Core; and evaporating the pharmaceutically acceptable solvent.

52. The method of claim 49, wherein said step (a) comprises granulating diphenhydramine with one or more pharmaceutically acceptable fillers and a polymeric binder.

53. The method of claim 49, wherein said coating of step (b) is applied by coacervation.

54. The method of claim 49, wherein said coating of step (b) comprises coating with a fluid bed coater.

55. The method of claim 49, wherein said compressing of step (d) is carried out using a rotary tablet press equipped with an external lubrication system to pre-lubricate the dies and punches.

56. The method of claim 49, wherein the at least one disintegrant, the at least one sugar alcohol and/or at least one saccharide are granulated to form rapidly dispersing granules prior to said mixing of step (c).

57. A method of treating the symptoms of one or more of allergic rhinitis, sinusitis, upper respiratory tract infections, motion sickness, Parkinson's disease, insomnia, and the common cold, comprising administering the composition of claim 29.

58. The method of treating the symptoms of one or more of allergic rhinitis, sinusitis, upper respiratory tract infections, motion sickness, Parkinson's disease, insomnia, and the common cold, comprising administering the composition of claim 36.

59. A method of managing pain comprising administering the composition of claim 37.
